(19) United States
(12) Patent Application Publication
(10) Pub. No.: US 2013/0317386 A1
(43) Pub. Date: Nov. 28, 2013

(54) BODY WEIGHT MANAGEMENT DEVICE HAVING FUNCTION OF PREDICTING BODY WEIGHT VARIATION

(71) Applicants: Teppei Sumino, Kusatsu-shi (JP); Takafumi Kamaya, Kyoto-shi (JP); Yoshitake Oshima, Kyoto-shi (JP)

(72) Inventors: Teppei Sumino, Kusatsu-shi (JP); Takafumi Kamaya, Kyoto-shi (JP); Yoshitake Oshima, Kyoto-shi (JP)

(73) Assignee: OMRON HEALTHCARE CO., LTD., Muko-shi, Kyoto (JP)

(21) Appl. No.: 13/983,849

(22) PCT Filed: Jan. 24, 2012
(86) PCT No.: PCT/JP2012/051394

§ 371 Date: Aug. 6, 2013

(30) Foreign Application Priority Data
Mar. 3, 2011 (JP) 2011-074688

(51) Int. Cl. A61B 5/053; A61B 5/00

(52) U.S. Cl. CPC A61B 5/0537; A61B 5/7275
USPC 600/547

(57) ABSTRACT

A body weight management device includes a measurement unit that measures a body impedance of a measurement subject, an impedance change detection unit that detects a temporal change in the measured body impedance, a variation amount computation unit that computes a body impedance variation amount in a unit period, and a determination unit that makes determination about a trend for subsequent changes in the measurement subject's body weight, using the body impedance variation in the unit period, and an output unit that outputs a result of the determination.

### TECHNICAL FIELD

This invention relates to body weight management devices, and particularly relates to body weight management devices having a function of predicting body weight variations.

### BACKGROUND ART

There is a demand for providing a function of predicting body weight variations in order to maintain a healthy body. For example, examples of said function include a method of measuring body weight values for a certain period and predicting body weight variations from fitted curves obtained from the measurement results, and the like.

Also, JP 2002-45346A (Patent Literature 1) describes a device that measures water content in a body to maintain a healthy body, determines the state of the body water content from the measurement results, and outputs the determination results in order to help understanding the pathology.

Also, JP 2002-112976A (Patent Literature 2) describes a body weight management device that comprehensively determines the condition of a subject's health by combining the state of body weight changes and the state of the body water content.

### CITATION LIST

### Patent Literature

Patent Literature 1: JP 2002-45346A
Patent Literature 2: JP 2002-112976A

### SUMMARY OF INVENTION

### Technical Problem

Here, it is known that body weight variations are related to body water content. Providing a function of predicting body weight variations from the relationship with body water content is desired for realizing accurate body weight management for the purpose of dieting or the like. However, the device described in JP 2002-45346A (Patent Literature 1) is not for predicting body weight variations. Also, the device described in JP 2002-112976A (Patent Literature 2) determines the condition of a subject's health using the state of the measured body water content and outputs the results thereof, but does not provide a function of predicting body weight variations using the state of body water content.

Therefore, it is an object of the present invention to provide a body weight management device that predicts and outputs trends for body weight changes.

### Solution to Problem

A body weight management device according to the present invention includes a measurement unit that measures a body impedance of a measurement subject, an impedance change detection unit that detects a temporal change in the measured body impedance, a variation amount computation unit that computes a body impedance variation amount in a unit period, based on the temporal change in the body impedance, a determination unit that makes determination about a trend for subsequent changes in a measurement subject's body weight, using the body impedance variation amount in the unit period, and an output unit that outputs a result of the determination.

### Advantageous Effects of Invention

According to the present invention, trends for body weight changes can be predicted and output.

### BRIEF DESCRIPTION OF DRAWINGS

FIG. 1 is a diagram of the external appearance of a body weight/body composition meter according to an embodiment of the present invention.

FIG. 2 is a diagram of the configuration of a body weight management system according to an embodiment of the present invention.

FIG. 3 is a diagram of the functional configuration, which is related to body weight management, of a body weight/body composition meter according to an embodiment of the present invention.

FIG. 4 is a flowchart of an overall process according to an embodiment of the present invention.

FIG. 5 is a flowchart of a recording/analysis process according to an embodiment of the present invention.

FIG. 6 is a flowchart of a weight loss determination process according to an embodiment of the present invention.

FIG. 7 is a flowchart of a rebound determination process according to an embodiment of the present invention.

FIG. 8 is a diagram illustrating examples of the stored content of a body weight storage unit according to an embodiment of the present invention.

FIG. 9 is a diagram illustrating examples of the stored content of an impedance storage unit according to an embodiment of the present invention.

FIG. 10 is a graph illustrating changes in the moving average values of body weights and impedances according to an embodiment of the present invention.

FIG. 11 is a diagram illustrating an example of display performed by a display unit according to an embodiment of the present invention.

FIG. 12 is a diagram illustrating an example of display performed by a display unit according to an embodiment of the present invention.

### DESCRIPTION OF EMBODIMENTS

Hereinafter, embodiments of the present invention will be described in detail with reference to the drawings. Note that identical or corresponding elements are given the same reference numerals in the drawings, and descriptions thereof will not be repeated.

In the present embodiment, “rebound” refers to a phenomenon in which a state in which a body weight continuously decreases successfully changes to a state in which the body weight increases. A “rebound period” refers to a period during which a state in which a body weight increases as a result of rebound continues.

Also, a “retention period” refers to a period in which a state in which a body weight does not change continues after a state in which a body weight successfully decreases.

In the present embodiment, a body weight/body composition meter capable of obtaining not only a body weight but also a given type of body composition information, such as a body fat percentage, by measuring a body impedance, is illustrated as an example of a body weight management device.

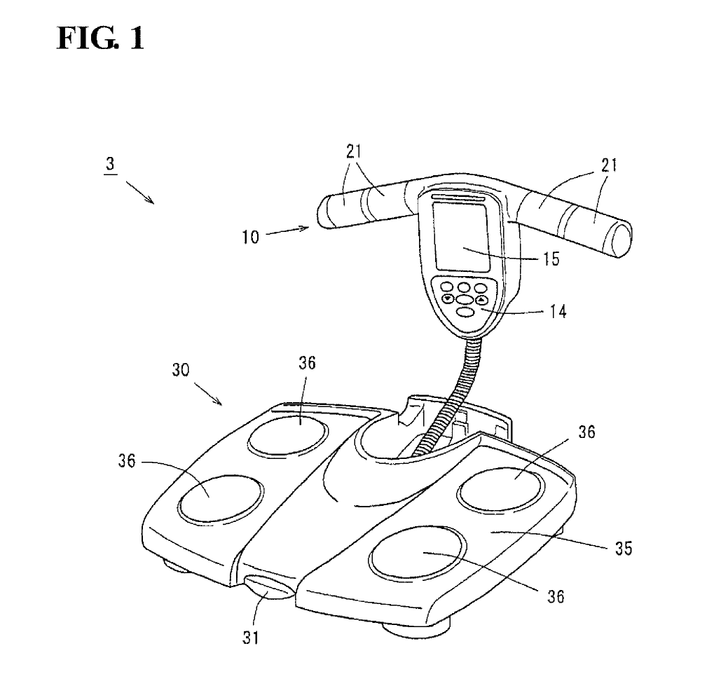

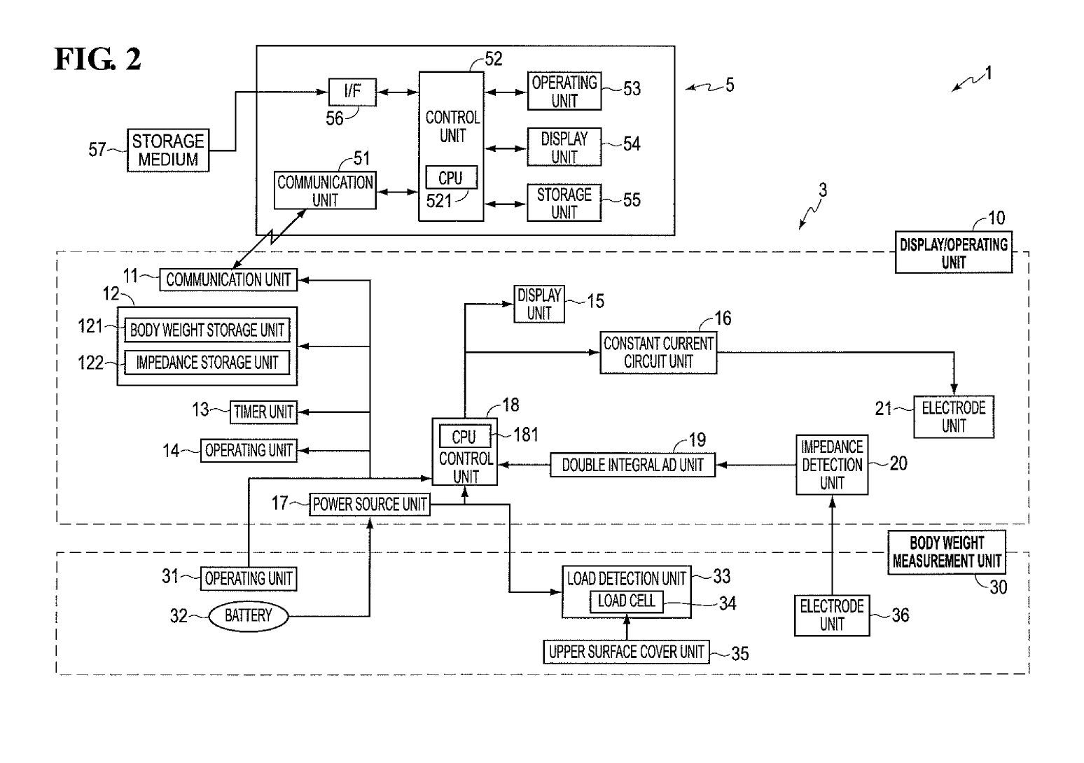

FIG. 1 is a diagram of the external appearance of a body weight/body composition meter 3 according to an embodiment of the present invention. FIG. 2 is a diagram of the configuration of a body weight management system 1 according to an embodiment of the present invention.

The body weight management system 1 shown in FIG. 2 includes the body weight/body composition meter 3 and a server (server computer) 5 that communicates with the body weight/body composition meter 3. To simplify the descriptions, FIG. 2 illustrates a single body weight/body composition meter 3 being connected to the server 5, but multiple body weight/body composition meters 3 may be connected. In FIG. 2, the body weight/body composition meter 3 and the server 5 communicate wirelessly or over wires. Note that the exchange of data between the body weight/body composition meter 3 and the server 5 is not limited to communications, and the exchange may take place via a storage medium.

As shown in FIG. 1, the body weight/body composition meter 3 includes a display/operating unit 10, which is a first housing member held by a measurement subject's (a user's) hand, and a body weight measurement unit 30, which is a second housing member onto which the measurement subject steps.

The display/operating unit 10 includes, as shown in FIG. 2, a communication unit 11, a storage unit 12, a timer unit 13, an operating unit 14, a display unit 15, a constant current circuit unit 16, a power source unit 17, a control unit 18 that includes a CPU (central processing unit) 181, a double integral AD (analog/digital) unit 19, an impedance detection unit 20, and electrode units 21.

The communication unit 11 is connected to the control unit 18, and communicates with the server 5 in accordance with a control signal from the control unit 18. Note that the communication unit 11 is not limited to communicating with the server 5; the communication unit 11 may communicate with any appropriate device, including another body information obtainment device such as a pedometer or the like, or a personal computer, mobile information terminal (a PDA (personal digital assistant), a mobile telephone, or the like), and so on.

The storage unit 12 includes an apparatus that can store information, such as a non-volatile memory, a hard disk, or the like. The storage unit 12 has information read out therefrom and written thereto in accordance with a control signal from the control unit 18, to which the storage unit 12 is connected. The storage unit 12 includes a body weight storage unit 121 for storing measured body weights, and an impedance storage unit 122 for storing measured (computed) impedance values.

The timer unit 13 is a device configured of a timer/counter that measures time such as the present day/time or the like, and outputs measured time to the control unit 18 as necessary.

The operating unit 14 includes multiple buttons/switches and the like (see FIG. 1) that are operated by being depressed or the like. By manipulating the operating unit 14, the measurement subject can input his/her personal information/body information, such as an ID (idenfier), sex, age, height, body weight, and so on. The inputted information is outputted to the control unit 18.

The display unit 15 is configured of a display device such as a liquid-crystal display (see FIG. 1), and displays images such as text, graphics, or the like in accordance with an image signal supplied from the control unit 18.

The constant current circuit unit 16 applies a high-frequency (AC) current supplied from the power source unit 17 to current application electrode units 21 in a single direction, under the control of the control unit 18.

The power source unit 17 supplies operational electricity to the respective elements, including the control unit 18. The control unit 18 is configured of a micon (a microcomputer) that includes the CPU 181, a ROM (read-only memory), and a RAM (random access memory) (not shown), and executes control operations and computational operations for the respective constituent elements in accordance with programs and various types of data stored in the ROM or the like.

These programs and data include programs and data for body weight management.

The double integral AD unit 19 is a double integral-type AD conversion unit. During operations, the double integral AD unit 19 converts an analog signal (a voltage signal) outputted from the impedance detection unit 20 into a digital signal and outputs that digital signal to the control unit 18.

The impedance detection unit 20 calculates a body impedance of the measurement subject based on a potential difference between electrode units 36 provided in the body weight measurement unit 30 and the electrode units 21 provided in the display/operating unit 10. Electrical characteristics resulting from the fact that a human body is constituted of highly conductive tissues (conductor: fat-free) and low conductive tissues (insulator: body fat), and the ratio between the two types of tissues reflect the body impedance values. Therefore, an electric current easily flows through muscle and body water as compared with fat and muscle and body water have low electrical resistance, and thus it can be understood that if the constitution of other tissues does not change, body water content is higher as a computed impedance value is lower whereas body water content is lower as a computed impedance value is higher.

The electrode units 21 are provided on the surfaces of grip portions (see FIG. 1) in the display/operating unit 10, which are held in the measurement subject's hand. The electrode units 21 apply the high-frequency (AC) current, supplied from the power source unit 17, to the palms of the measurement subject's hands that are gripping the grip portions in order to calculate the body impedance.

The body weight measurement unit 30 includes an operating unit 31, a battery 32, a load detection unit 33, and the electrode units 36. The operating unit 31 functions as an input switch that is manipulated in order to switch the power on or off, and when the operating unit 31 is manipulated, an input signal is outputted to the control unit 18 in response to that manipulation.

The battery 32 supplies power to the respective elements, and in particular, to the power source unit 17.

The load detection unit 33 has multiple load cells 34 provided therein. The load detection unit 33 measures the body weight of the measurement subject that has stepped onto an upper surface cover unit 35 (see FIG. 1) that also serves as an upper surface cover of the housing member. The measured body weight is outputted to the double integral AD unit 19.

The electrode units 36 are provided in the surface of the upper surface area of the body weight measurement unit 30 (see FIG. 1) onto which the measurement subject steps, and serve as current measurement electrodes that detect a current that flows from the soles of the measurement subject's feet in order to calculate the body impedance. The electrode units 36 include four electrodes that make contact with the left toe side, the left heel side, the right toe side, and the right heel side of the measurement subject's feet.

Each of the load cells 34 in the load detection unit 33 is disposed so as to be capable of measuring a load placed on the upper surface area of the body weight measurement unit 30, and here, are disposed below the respective electrodes in the electrode units 36. Accordingly, both the body impedance and the body weight can be measured when the measurement subject steps upon the upper surface area.

During body weight measurement, a load produced by the measurement subject's body weight is exerted on the load cells 34. Each of the load cells 34 is configured of a bending member, formed of a metal member that deforms in response to a load exerted thereon, and a strain gauge that is applied to the bending member. When the bending member bends, the strain gauge extends/contracts, and a resistance value changes in accordance with the extension/contraction of the strain gauge; the change in resistance is then derived as a load signal output. Accordingly, in the case where the measurement subject has stepped onto the upper surface area and both feet have been placed on the load cells 34, the bending member will bend clue to the measurement subject's body weight that has been applied to the load cells 34, and the body weight will be measured as a change in the aforementioned load signal output.

Although the load cells 34 are used in the present embodiment as load sensors for detecting a load, it should be noted that a sensor that employs, for example, springs, a piezoelectric film, or the like, a compression element, a displacement sensor, or the like may be used as long as that element is capable of detecting the amount of a force applied to the upper surface area.

The server 5 includes a communication unit 51, a control unit 52, an operating unit 53, a display unit 54, and a storage unit 55. The control unit 53 is configured of a computer having a CPU 521, a ROM, and a RAM. Furthermore, the server 5 includes an I/F (interface) 56 on which a storage medium 57 such as a CD-ROM (compact-disc read only memory) or the like is to be detachably mounted and that accesses (reads out and writes to) the mounted storage medium 57 under the control of the CPU 521.

The communication unit 51 exchanges data with the body weight/body composition meter 3 under the control of the control unit 52. The CPU 521 of the control unit 52 controls the operations of the respective elements and executes various types of computations in accordance with programs and data stored in the ROM or the like.

The operating unit 53 includes a keyboard, a mouse, or the like. Signals inputted as a result of operations performed by an operator are outputted to the control unit 52.

The display unit 54 corresponds to a liquid-crystal display, a CRT (cathode ray tube) display, or the like. The display unit 54 displays images such as graphics, text, or the like in accordance with a control signal supplied from the control unit 52.

The storage unit 55 corresponds to a fixed storage device such as a hard disk, or a recording medium that can be read by the computer that includes the CPU 521, such as a flexible disk, a CD-ROM (compact disk read-only memory), a ROM (read-only memory), a RAM (random access memory), a memory card, and so on.

The storage unit 55 stores various types of data related to the measurement subject, such as data measured by the body weight/body composition meter 3 (body composition information, body weight data, measurement day/time data, and so on), and personal information such as the measurement subject's name (ID), address, and so on.

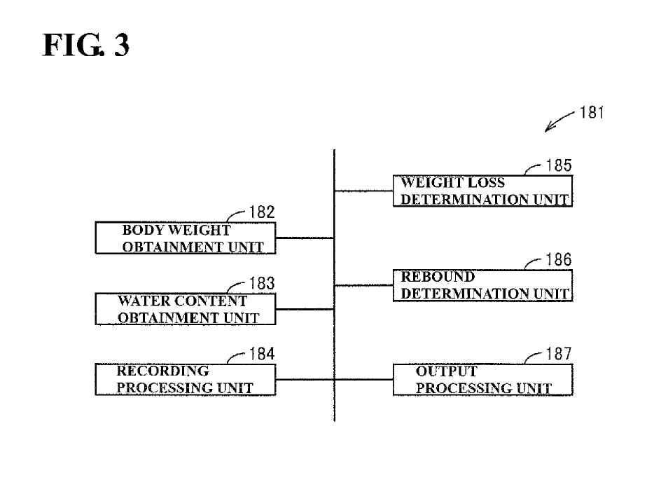

FIG. 3 is a diagram of the functional configuration, which is related to body weight management, of a body weight/body composition meter 3 according to an embodiment of the present invention. As shown in FIG. 3, the CPU 181 includes a body weight obtainment unit 182, a water content obtainment unit 183, a recording processing unit 184, a weight loss determination unit 185, a rebound determination unit 186, and an output processing unit 187. These elements are realized by programs executed by the CPU 181 or the combination of programs and circuits. These programs are stored in advance in the ROM (not shown) of the control unit 18. The functions of the respective elements are realized by the CPU 181 reading out the programs from the ROM and executing the commands in the read-out programs.

The body weight obtainment unit 182 computes a body weight based on a signal output from the load detection unit 33.

The water content obtainment unit 183 obtains a body impedance value output from the double integral AD unit 19 as body water content.

The recording processing unit 184 respectively stores, in the body weight storage unit 121 and the impedance storage unit 122, the body weight and the body water content of the measurement subject obtained by the body weight obtainment unit 182 and the water content obtainment unit 183. In the present embodiment, the body weight and the body impedance are measured at the same time as described above, and thus the recording processing unit 184 associates the body weight and the body impedance with measurement time measured by the timer unit 13 and stores the associated information in the storage unit 12. Also, the recording processing unit 184 reads out from the storage unit 12 the associated body weight, body impedance and measurement time when reading out the data from the storage unit 12.

The weight loss determination unit 185 determines whether or not the body is in a weight loss period in which the body weight is continuously decreasing successfully based on the body weight variations.

The rebound determination unit 186 determines whether the body weight has rebounded or is in a rebound period.

The output processing unit 187 performs processes for displaying information on the display unit 15 or for sending information to the server 5 via the communication unit 11.

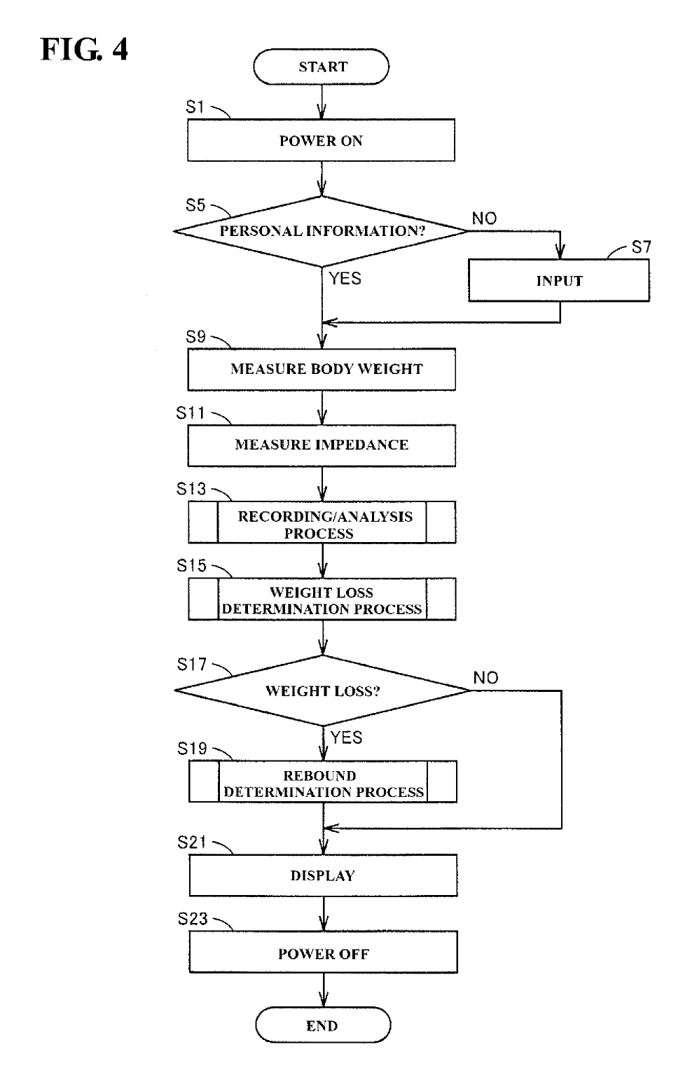

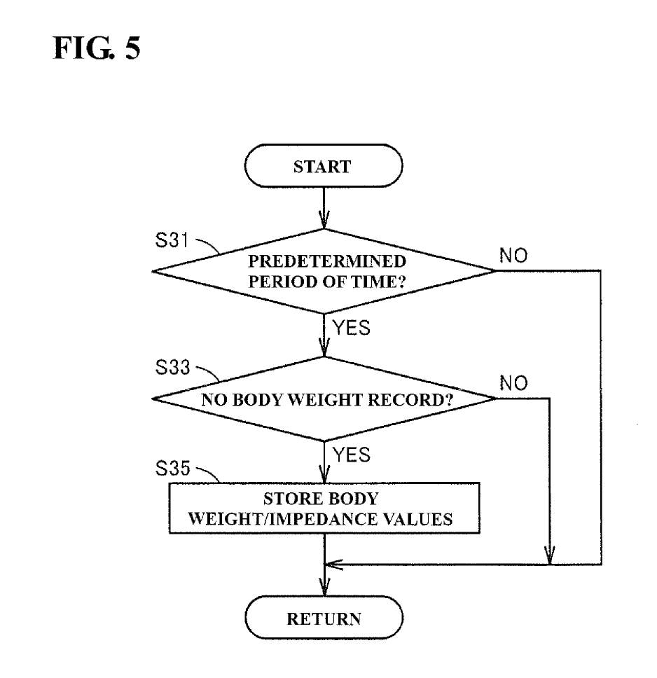

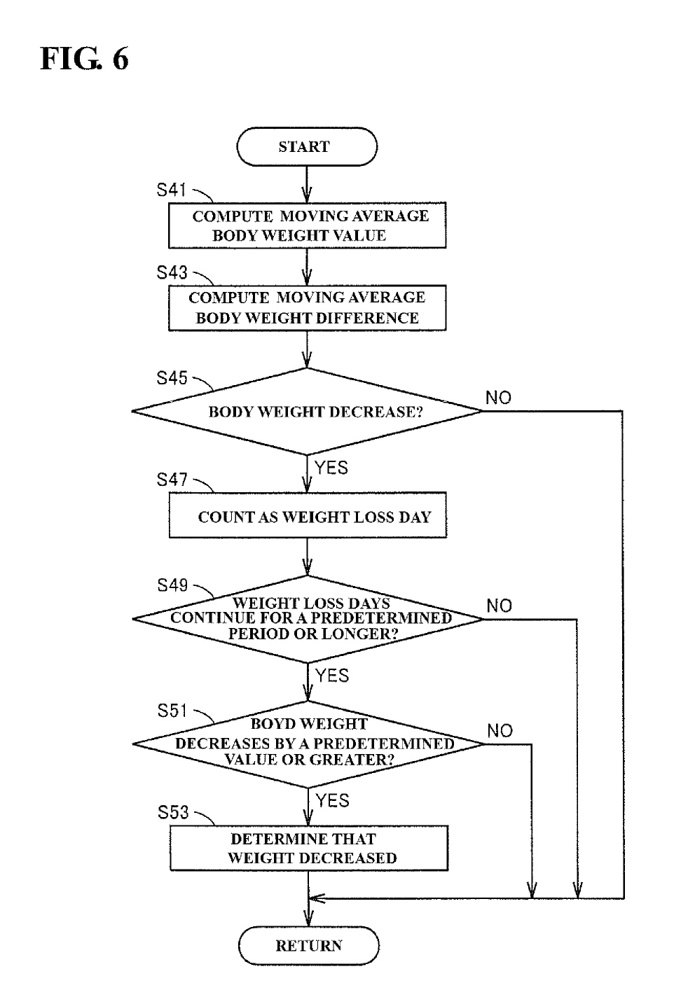

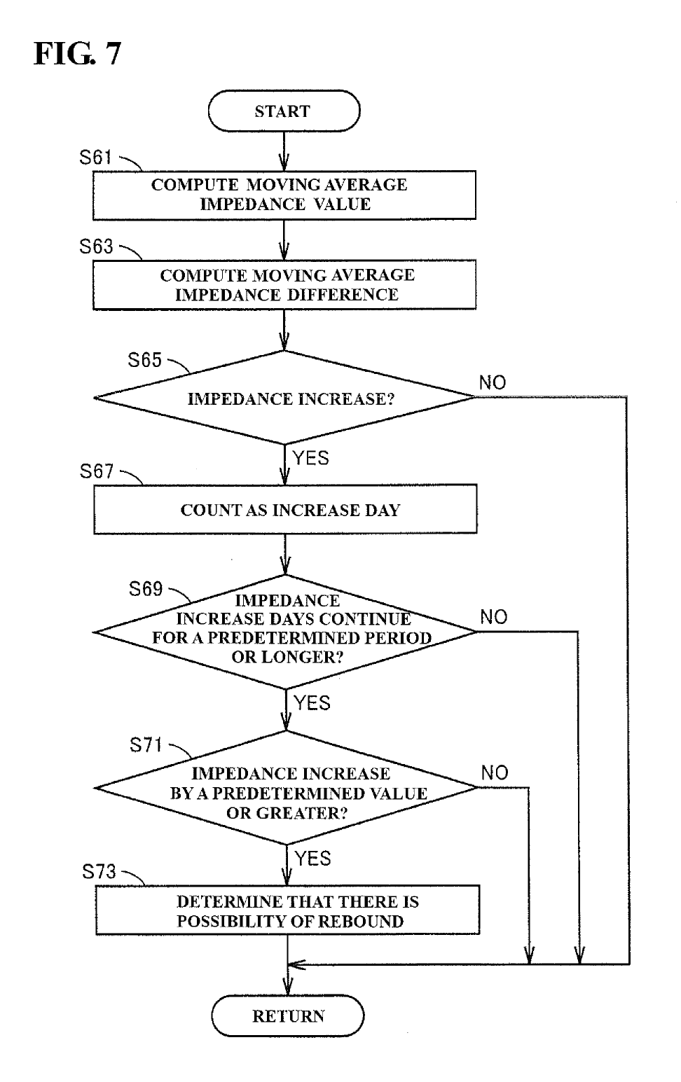

FIG. 4 is a flowchart of an overall process according to an embodiment of the present invention. FIG. 5 is a flowchart of a recording/analysis process according to an embodiment of the present invention. FIG. 6 is a flowchart of a weight loss determination process according to an embodiment of the present invention. FIG. 7 is a flowchart of a rebound determination process according to an embodiment of the present invention.

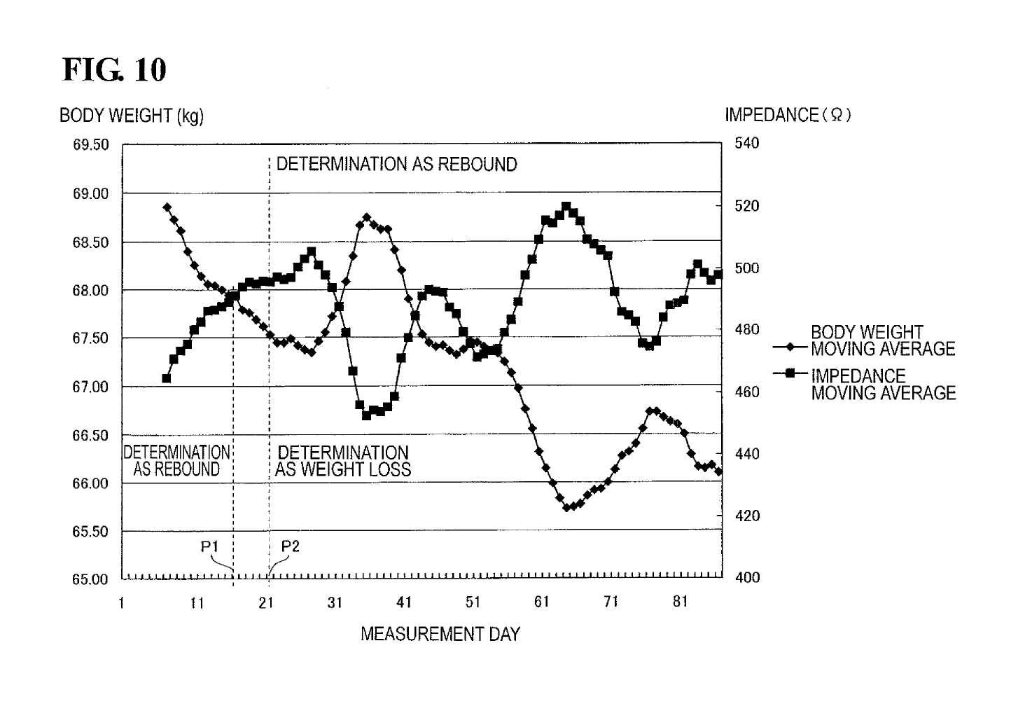

FIG. 10 is a graph illustrating changes in the moving average values of body weights and impedances according to an embodiment of the present invention. The horizontal axis of the graph indicates each day of body weight management for a predetermined period as a measurement day. Also, the vertical axis indicates the measured body weight (unit: kg) and impedance (unit: Ω).

Programs according to these flowcharts are stored in advance in a predetermined region of the storage unit 12, and the processes are realized by the CPU 181 reading out the programs from the storage region and executing the read-out programs.

Here, it is assumed that the measurement subject uses the body weight/body composition meter 3 for the purpose of dieting, that is, losing weight. In the present embodiment, a body weight measured everyday in a predetermined period of time is used in order to provide appropriate support information for body weight management. It is preferable that a body weight used is measured in a predetermined period of time after he/she wakes which is the time in which the body weight of the measurement subject is the lowest in one day. The information in the predetermined period of time is assumed to be stored in the predetermined region of the storage unit 12 in advance. Note that a message “please measure body weight in a predetermined period of time (for example, from XX a.m. to YY a.m.) after waking up and before eating and after going to lavatory for body weight management” is displayed on the display unit 15 for the measurement subject. Accordingly, the measurement subject forms a habit to measure his/her body weight in a predetermined period of time after waking up.

Overall Process

As shown in FIG. 4, the measurement subject manipulates the operating unit 31 and turns on a power source in order to start measurement. The CPU 181 inputs an instruction of turning on the power source (step S1), based on a signal input from the operating unit 31. Accordingly, power is supplied to the respective elements from the power source unit 17.

The CPU 181 determines whether personal information (information such as height, sex, age and so on) has been already stored in the storage unit 12. These pieces of information are the information required for computing a body composition. If it is determined that the personal information has been already stored (YES in step S5), the process moves to step S9. If it is determined that the personal information has not yet been stored, these pieces of information input by the measurement subject manipulating the operating unit 14 are stored in the predetermined region of the storage unit 12 (step S7), and the process moves to step S9.

Next, a measurement process is carried out. At this time, the measurement subject is in a state in which he/she is standing on the upper surface cover unit 35 (see FIG. 1). The body weight obtainment unit 182 obtains the body weight and the water content obtainment unit 183 obtains the body impedance (steps S9 and S11).

Next, a recording/analysis process (step S13) which will be described later is carried out. In the recording/analysis process, the obtained body weight and impedance are associated with the data on measurement day/time indicating date/time based on the time sent from the timer unit 13 and are stored respectively in the body weight storage unit 121 and the impedance storage unit 122.

Next, the weight loss determination unit 185 carries out a weight loss determination process which will be described later (step S15), based on the data read out from the body weight storage unit 121.

Based on the output from the weight loss determination unit 185, when it is determined that the body is not in a weight loss period (NO in step S17), the process moves to step S21 whereas when it is determined that the body is in a weight loss period (YES in step S17), a rebound determination process (step S19) which will be described later is carried out by the rebound determination unit 186.

The results of the determination processes are output by the output processing unit 187 (step S21). For example, the results are displayed on the display unit 15, sent to the server 5 via the communication unit 11, or stored in a predetermined region of the storage unit 12.

After outputting the determination results, the CPU 181 inputs an instruction given by a user manipulating the operating unit 31. In other words, the measurement subject turns off the power source via the operating unit 31 in the case of ending the measurement, and thus the CPU 181 inputs an instruction of turning off the power source given from the operating unit 31 and controls the power source unit 17 so as to disconnect power supply to the respective elements (step S23), based on the instruction of turning off the power source. Accordingly, a series of processes ends.

Recording/Analysis Process

The recording/analysis process (step S13) will now be described with reference to FIG. 5. The CPU 181 determines whether or not the present time corresponds to a predetermined period of time on the basis of time data based on the time sent from the timer unit 13 (step S31). If it is determined that the present time does not correspond to the predetermined period of time (NO in step S31), data on the measured body weight and impedance is discarded without being stored in the storage unit 12, and the recording/analysis process ends.

Also, if it is determined that the present time corresponds to the predetermined period of time (YES in step S31), the CPU 181 determines whether or not the body weight data associated with the predetermined period of time is stored in the body weight storage unit 121 (step S33). If it is determined that the body weight data has been already stored (YES in step S33), the measured body weight and impedance are discarded without being stored in the storage unit 12, and the recording/analysis process ends, whereas if it is determined that the body weight data has not yet been stored (NO in step S33), the obtained body weight, impedance and measurement time are associated with one another and respectively stored in the body weight storage unit 121 and the impedance storage unit 122 of the storage unit 12 (step S35).

Here, although it is assumed that body weight data and impedance data that are not stored in the storage unit 12 are discarded, such data may be stored in another storage region of the storage unit 12. Alternatively, such data may be displayed on the display unit 15, instead of being stored in the storage unit 12.

Examples of Stored Content

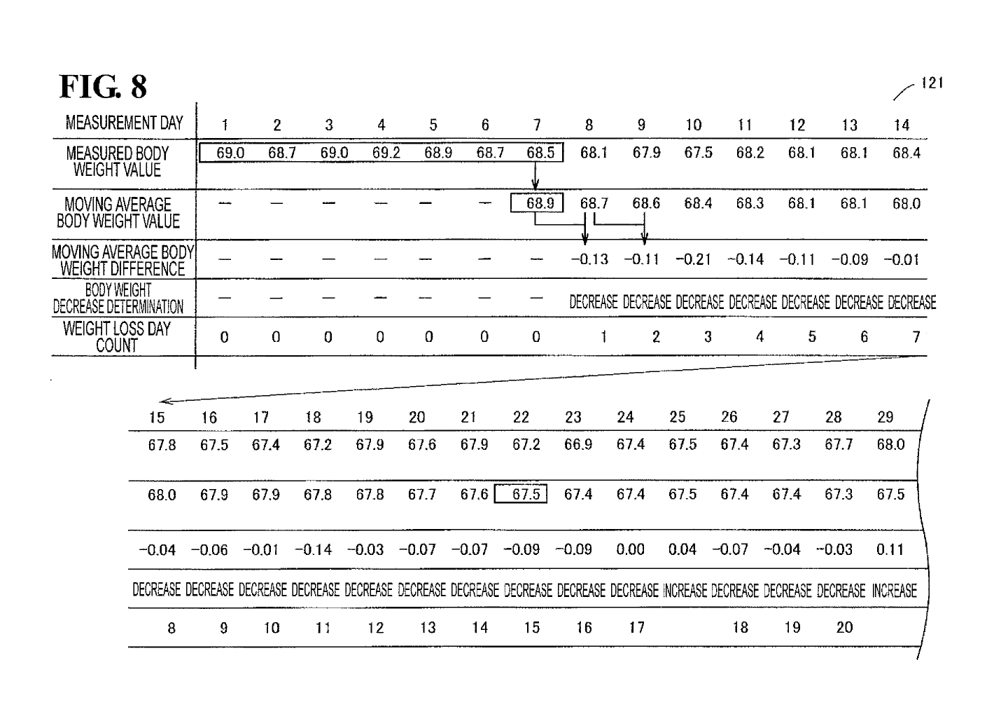

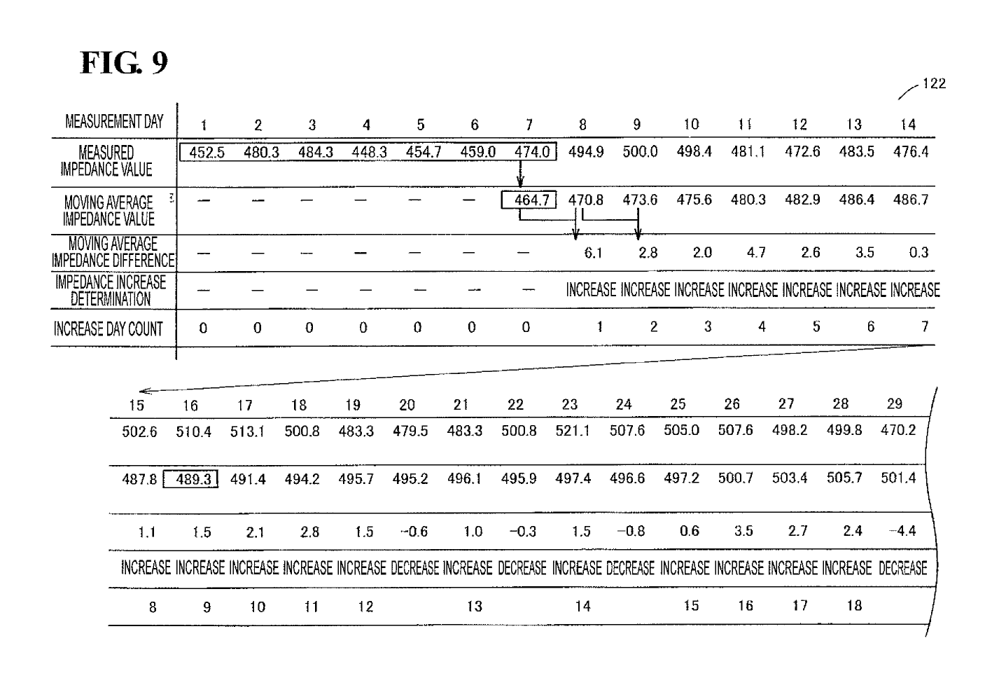

FIG. 8 is a diagram illustrating examples of the stored content of the body weight storage unit 121 according to an embodiment of the present invention. FIG. 9 is a diagram illustrating examples of the stored content of the impedance storage unit 122 according to an embodiment of the present invention. As shown in FIGS. 8 and 9, a body weight management period is assumed for dieting through the recording/analysis process. Here, it is assumed that a starting day of the body weight management period is the first day, and the current day is the twenty ninth day. FIGS. 8 and 9 show a state in which values such as measured body weight values, measured impedance values and the like are stored from the first day to that day (twenty ninth day) in the body weight management period.

Weight Loss Determination Process

Next, the weight loss determination process (step S15) will now be described based on the flowchart of FIG. 6 with reference to FIG. 10.

The weight loss determination unit 185 computes a simple moving average with regard to the measured body weight values of the body weigh storage unit 121, and determines whether or not the body weight is in a period of loss using the computed movement averages.

Here, moving averages for the latest days, for example, for the last seven days, are computed. Thus, an average from the first day to the seventh day in the body weight management period is firstly computed on the seventh day, an average from the second day to the eighth day is computed on the eighth day, and an average from the third day to the ninth day is computed on the ninth day. Subsequently, averages are computed with regard to seven days in a one day shifting manner. As a result, moving average body weight values are stored in the body weight storage unit 121, as shown in FIG. 8.

The weight loss determination unit 185 computes, on the twenty ninth day, the average of the measured body weights from the twenty third day to that day (twenty ninth day), and stores the computed result as the moving average body weight value (67.5 kg in FIG. 8) on the twenty ninth day (step S41).

The weight loss determination unit 185 computes a moving average body weight difference which is a difference between the moving average body weight value computed in step S41 and the moving average body weight value (67.3 kg in FIG. 8) computed on the previous day (the day before), and stores the computed moving average body weight difference as a moving average body weight difference (0.11 kg in FIG. 8) on the twenty ninth day (step S43).

The weight loss determination unit 185 determines whether or not the body weight decreases based on the computed moving average body weight difference (step S45). Specifically, if the moving average body weight difference is a negative value, it is determined that the body weight decreases (YES in step S45). On the other hand, if the moving average body weight difference is 0 or a positive value, it is determined that the body weight does not decrease (NO in step S45), and “increase” is stored as data on the body weight decrease determination on the twenty ninth day, and the weight loss determination process ends.

In the case where it is determined that the body weight decreases (YES in step S45), “decrease” is stored as data on the body weight decrease determination on the twenty ninth day. The weight loss determination unit 185 stores a value obtained by incrementing a weight loss day count value by 1 as a weight loss day count on the twenty ninth day (step S47). The weight loss day count is not stored in FIG. 8 because the body weight is determined as “increase” on the twenty ninth day.

The weight loss determination unit 185 determines whether or not weight loss days continue for a predetermined period or longer (step S49). For example, in the case where it is determined that data on the body weight decrease determination stored with regard to the last ten days does not indicate “decrease” (NO in step S49), the determination result indicating that the body is not in a weight loss period is output, and the process ends.

On the other hand, in the case where it is determined that data on the body weight decrease determination stored with regard to the last ten days indicates “decrease” (YES in step S49), the process moves to step S51. According to FIG. 8, it is determined that “decrease” continues for a predetermined period (ten days) on the seventeenth day. Thereafter, the weight loss determination unit 185 next determines whether the body weight decreases by a predetermined value or greater (step S51). Specifically, in the case where it is determined that the moving average body weight value on that day is less than or equal to a value obtained by subtracting 2% from a reference value where a moving average body weight value (moving average body weight value on the seventh day in FIG. 8) at a point in time when the weight loss day count value of the body weight storage unit 121 changes from 0 to 1 is denoted as the reference, it is determined that the body weight decreases by a predetermined value or greater. For example, if the moving average body weight value (68.9 kg) on the seventh day is used, it is determined that the body weight decreases by a predetermined value or greater (YES in step S51), for example, on the twenty second day on which a moving average body weight value indicating a weight loss by 68.9 kg×0.02=1.4 kg is computed. This determination corresponds to a weight loss determination on the twenty second day indicated by a point P2 of FIG. 10.

If it is determined that the body weight does not decrease by a predetermined value or greater (NO in step S51), the weight loss determination unit 185 outputs a determination result indicating that the body is not in a weight loss period, and ends the process. On the other hand, if it is determined that the body weight decreases by a predetermined value or greater (YES in step S51), the weight loss determination unit 185 outputs a determination result indicating that the body is in a weight loss period (step S53), and ends the process.

Rebound Determination Process

Next, the rebound determination process (step S19) will now be described with reference to FIG. 7.

The rebound determination unit 186 computes a simple moving average with regard to the measured impedance values of the impedance storage unit 122, and determines whether or not the impedance is in an increasing period, using the computed moving averages. As described above, when the impedance is in an increasing trend, the body water content is in a decreasing period, whereas when the impedance is in a decreasing trend, the body water content is in an increasing period.

Here, similarly to the body weight, moving averages for the latest days, for example, for the last seven days, are computed. Therefore, as shown in FIG. 9, moving average impedance values are stored in the impedance storage unit 122.

The rebound determination unit 186 computes, on the twenty ninth day, an average of measured impedance values from the twenty third day to that day (twenty ninth day), and stores the computation result as a moving average impedance value (501.4Ω in FIG. 9) on the twenty ninth day (step S61).

The rebound determination unit 186 computes a moving average impedance difference which is a difference between the moving average impedance value computed in step S61 and the moving average impedance value (505.7Ω in FIG. 9) on the previous day (the day before), and stores the computed moving average impedance difference as a moving average impedance difference (−4.4Ω in FIG. 9) on the twenty ninth day (step S63).

The rebound determination unit 186 determines whether or not the impedance increases based on the computed moving average impedance difference (step S65). Specifically, if the moving average impedance difference is a negative value, it is determined that the impedance decreases (NO in step S65), and if it is 0 or a positive value, it is determined that the impedance increases (YES in step S65). “Decrease” is stored as data on the impedance increase determination on the twenty ninth day, the determination result indicating that there is no possibility of rebound is output, and the rebound determination process ends.

In the case where it is determined that the impedance increases (YES in step S65), “increase” is stored as data on the impedance increase determination on the twenty ninth day. The rebound determination unit 186 then stores a value obtained by incrementing an increase day count value by 1 as an increase day count on the twenty ninth day (step S67). The increase day count is not stored in FIG. 9 because the impedance is determined as “decrease” on the twenty ninth day.

The rebound determination unit 186 determines whether or not increase days continue for a predetermined period or longer (step S69). For example, in the case where it is determined that the impedance increase determinations are not stored as “increase” with regard to the last ten days (NO in step S69), the determination result indicating that there is no possibility of rebound is output, and the process ends.

On the other hand, in the case where it is determined that the impedance increase determinations are stored as “increase” with regard to the last ten days (YES in step S69), the process moves to step S71. According to FIG. 9, it is determined that “increase” continues for a predetermined period (ten days) on the seventeenth day.

Thereafter, the rebound determination unit 186 next determines whether or not the impedance increases by a predetermined value or greater (step S71). Specifically, in the case where it is determined that the moving average impedance value on that day is greater than or equal to a value obtained by adding 5% to a reference value where a moving average impedance value (moving average impedance value on the seventh day in FIG. 9) at a point in time when the increase day count value of the impedance storage unit 122 changes from 0 to 1 is denoted as the reference, it is determined that the impedance increases by a predetermined value or greater. For example, if the moving average impedance value (464.7Ω) on the seventh day is used, it is determined that the impedance increases by a predetermined value or greater (YES in step S71), for example, on the sixteenth day on which a moving average impedance value indicating an increase by 464.7Ω×0.05=23.2Ω is computed. This determination corresponds to a determination indicating that there is the possibility of rebound on the sixteenth day indicated by a point P1 of FIG. 10.

If it is determined that the impedance does not increase by a predetermined value or greater (NO in step S71), the rebound determination unit 186 outputs a determination result indicating that there is no possibility of rebound, and ends the process. On the other hand, if it is determined that the impedance increases by a predetermined value or greater (YES in step S71), the rebound determination unit 186 outputs a determination result indicating that there is the possibility of rebound (step S73), and ends the process.

Also, the determination indicating that there is the possibility of rebound is also made on the twenty second day indicated by the point P2 of FIG. 10.

Referring to FIG. 10, according to the aforementioned determination processes, the determination indicating that there is the possibility of rebound is not output (step S21) because even though it is determined that there is the possibility of rebound on the sixteenth day as indicated by the point P1, it is determined that the body is not in a weight loss period. On the other hand, the determination indicating that there is the possibility of rebound is output (step S21) on the twenty second day as indicated by the point P2 because it is determined that there is the possibility of rebound and the body is in the weight loss period.

In this manner, the moving average impedance difference for a predetermined period is computed as a variation difference (slope) of body water content, trends for changes indicating whether the body weight of the measurement subject will be in a retention period or a rebound period thereafter based on the computed variation difference is predicted, and the measurement subject is notified of the result.

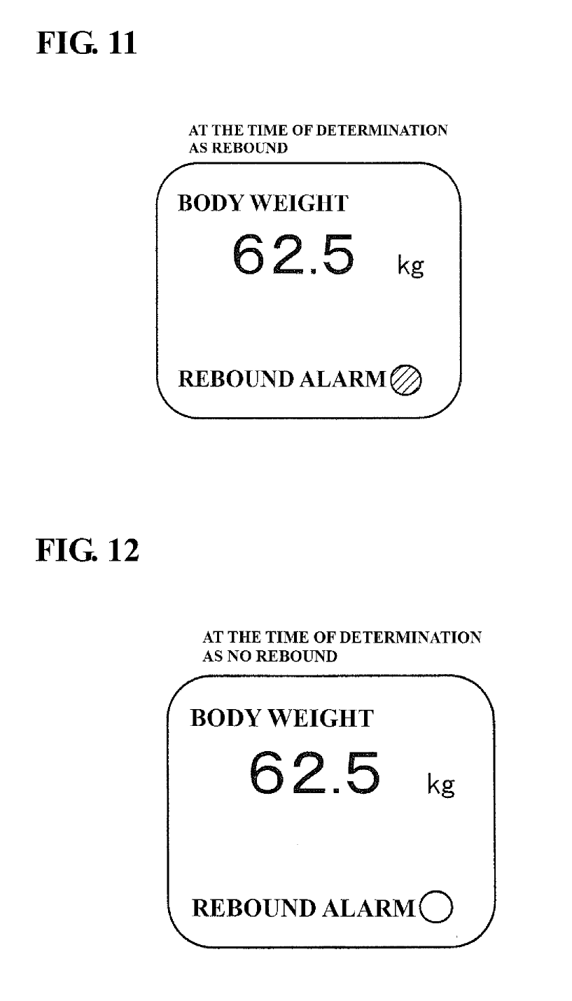

FIGS. 11 and 12 are diagrams illustrating examples of display performed by the display unit 15 according to an embodiment of the present invention. FIG. 11 shows a display screen that gives a notification indicating that there is the possibility of rebound at the point P2, and FIG. 12 shows a display screen that gives a notification indicating that there is no possibility of rebound at the point P1.

An alarm indicating that there is the possibility of rebound is given as a notification on the screen of FIG. 11. The degree of success in the current dieting (body weight decreasing successfully) may also be displayed on the screen of FIG. 11. Accordingly, advice indicating that there is the possibility of rebound even though the body weight successfully decreases can be given. At this time, a configuration may be adopted in which the measurement subject easily maintains motivation for body weight management by outputting a message indicating “predicted rebound is caused by body water content and weight loss is successful”.

Also, a configuration may be adopted in which for each measurement subject, a pattern analysis is performed on data on impedances measured for a long period that is stored in the impedance storage unit 122, average duration of a rebound period is computed and the computed average duration is displayed on the screen of FIG. 11 along with the possibility of rebound.

Also, a configuration may be adopted in which an average grace period from the day on which the possibility of rebound is determined to the day on which rebound is actually measured, that is, the day on which the body weight stops decreasing (or starts increasing), is computed from the result of analyzing body weight data that is measured for a long period and stored in the body weight storage unit 121 and the result of analyzing impedance data that is measured for a long period and stored in the impedance storage unit 122, and is displayed on the screen of FIG. 11.

Although a notification is given using display here, a notification may be given by audio instead of display. Also, the result determined by the body weight/body composition meter 3 may be sent to the server 5, and may be output by the server 5 (displayed, output as audio, and stored in the storage unit 55).

Another Method of Estimating Body Water Content

Although in the aforementioned embodiments, it is assumed that the body water content obtainment unit 183 deals with the measured body impedance variations as body water content variations based on a relationship that the body impedance decreases as the body water content increases, the body water content obtainment unit 183 may compute the body water content. For example, the computation method of JP 2002-112976A (Patent Literature 2) described above can be used.

In other words, an intracellular fluid weight ICw and an extracellular fluid weight ECw are computed based on parameter Re and Ri computed from the measured body impedance, the height (H) input in step S7 as personal information, and the measured body weight (W), and body water content TBw=ICw+ECw is computed.

ICw=Ki1×H2/Ri+Ki2×W+Ki3

ECw=Ke1×H2/Re+Ke2×W+Ke3

TBw=ICw+ECw

where Ki1, Ki2, Ki3, Ke1, Ke2 and Ke3 are predetermined coefficients. Also, the method of computing the parameters Re and Ri from the measured body impedance is described in detail in JP 2002-112976A (Patent Literature 2), and thus description thereof is not repeated.

The water content obtainment unit 183 may compute the body water content TBw using the body impedance through the above-described equations, and the rebound determination unit 186 may determine whether there is the possibility of rebound based on variations of the computed body water content TBw.

In the present embodiment, because the increase or decrease of body water content has a greater effect on the factor in the body weight variations in a short period than variations of muscle mass or fat mass have, determination is focused on the fact that when the body weight increases thereafter, the body water content increases before body weight increases, and when the body weight decreases, the body water content decreases before body weight decreases. In the case where even if the body weight successfully decreases, a prediction is given that the body weight is likely to increase after a few days, that is, the body will be in the body weight retention period or rebound period, based on a computation result obtained by computing a variation difference (positive (increase) slope, negative (decrease) slope, and the magnitude thereof) of body water contents for a predetermined period, the measurement subject is notified thereof. Accordingly, the measurement subject can improve or maintain motivation for body weight management such as dieting.

Also, the results determined by the weight loss determination unit 185 or the rebound determination unit 186 may only be output. In other words, only the result of the determination about whether or not the body is in the weight loss period, or the result of determination about whether or not the body water content tends to increase may be output. In this manner, a mode in which only the result of determination made by the weight loss determination unit 185 or the rebound determination unit 186 is output, and a mode in which a result obtained by combining the results made by the weight loss determination unit 185 or the rebound determination unit 186 (a result of determination about whether there is the possibility of rebound) is output may be switched as output modes by the measurement subject manipulating the operating unit 14.

### Other Embodiments

The server 5 may include the body weight management function performed by the body weight/body composition meter 3 of the aforementioned embodiment.

The server 5 receives data on the body weight and impedance measured by the body weight/body composition meter 3 from the body weight/body composition meter 3, determines whether the body is in the weight loss period or there is the possibility of rebound based on the received data, outputs the determination result, and the like. Therefore, it is possible to cause the server 5 to determine whether the body is in the weight loss period or there is the possibility of rebound according to the present embodiment, instead of the body weight/body composition meter 3.

The server 5 is typically configured of a general purpose computer. The programs according to the aforementioned functions executed by the server 5 are stored in the storage medium 57 such as the CD-ROM (compact disc-read only memory) or the like and are distributed therefrom. The programs stored in the storage medium 57 are read out by the I/F 56 having a function corresponding to a CD-ROM (compact disk-read only memory) drive, and are stored in a hard disk (not shown) which is a portion of the storage unit 55 in the server 5. Alternatively, a configuration may be adopted in which the programs are downloaded from an upstream host computer or the like through a network.

Note that the embodiments disclosed above are to be understood as being in all ways exemplary and in no way limiting. The scope of the present invention is defined not by the aforementioned descriptions but by the scope of the appended claims, and all changes that fall within the same essential spirit as the scope of the claims are intended to be included therein as well.

### REFERENCE SIGNS LIST

1 body weight management system
3 body weight/body composition meter
5 server
10 display/operating unit
13 timer unit
15 display unit
20 impedance detection unit
30 body weight measurement unit
57 storage medium
121 body weight storage unit
122 impedance storage unit
182 body weight obtainment unit
183 water content obtainment unit
184 recording processing unit
185 weight loss determination unit
186 rebound determination unit
187 output processing unit

1. A body weight management device comprising:
a measurement unit that measures a body impedance of a measurement subject;
an impedance change detection unit that detects a temporal change in the measured body impedance;
a variation amount computation unit that computes a body impedance variation amount in a unit period, based on the temporal change in the body impedance;
a determination unit that makes determination about a trend for subsequent changes in a measurement subject's body weight, using the body impedance variation amount in the unit period; and
an output unit that outputs a result of the determination.

2. The body weight management device according to claim 1, further comprising:
a body weight obtainment unit that obtains body weight data of the measurement subject;
a body weight change detection unit that detects a temporal change in the body weight, based on the obtained body weight data; and
a body weight variation amount computation unit that computes the body weight variation amount in the unit period, based on the temporal change in the body weight,
wherein the determination unit makes determination about a trend for subsequent changes in the measurement subject's body weight, from a relationship between
the body impedance variation amount in the unit period and the body weight variation amount in the unit period.

3. The body weight management device according to claim 2, wherein the determination unit makes determination about a trend for subsequent changes in the measurement subject's body weight, from a relationship between an increase in the body impedance in the unit period and a decrease in the body weight in the unit period.

4. The body weight management device according to claim 3, wherein the determination unit makes determination about a trend for subsequent changes in the measurement subject's body weight, from a relationship between a decrease in the body weight in a case where the body weight continuously decreases in the unit period and an increase in the body impedance in the unit period.

5. The body weight management device according to claim 3, wherein the determination unit makes determination about a trend for subsequent changes in the measurement subject's body weight, from a relationship between an increase in the body impedance in a case where the body impedance continuously increases in the unit period and a decrease in the body weight in a case where the body weight continuously decreases in the unit period.

6. The body weight management device according to claim 1, which computes body water content of the measurement subject, using the measured body impedance.

7. The body weight management device according to claim 1, further comprising:
a water change detection unit that detects a temporal change in body water content of the measurement subject, using the measured body impedance; and
a water variation amount computation unit that computes a water variation amount of the water content in a unit period, based on the temporal change in the water content,
wherein the device makes determination about a trend for subsequent changes in the measurement subject's body weight, using the water variation amount in the unit period.

8. A body weight management device comprising:
an obtainment unit that obtains a body impedance of a measurement subject that is to be measured;
a detection unit that detects a temporal change in the obtained body impedance;
a variation amount computation unit that computes a body impedance variation amount in a unit period, based on the temporal change in the body impedance;
a determination unit that makes determination about a trend for subsequent changes in the measurement subject's body weight, using the body impedance variation amount in the unit period; and
an output unit that outputs a result of the determination.
